(19) United States
(12) Patent Application Publication
(10) Pub. No.: US 2024/0233927 A1
(43) Pub. Date: Jul. 11, 2024

(54) SYSTEMS AND METHODS FOR AN INTERACTIVE AND DYNAMIC INTERFACE

(71) Applicant: VIZIENT, INC., Irving, TX (US)

(72) Inventors: Allan S. Frankel, Concord, MA (US); Michael W. Leonard, Evergreen, CO (US); Terri Christensen Frankel, Concord, MA (US); Joshua Proulx, Lehi, UT (US); Daniel Joseph Cramer, Murray, UT (US); John D. Duce, Lehi, UT (US)

(21) Appl. No.: 18/409,497

(22) Filed: Jan. 10, 2024

Related U.S. Application Data
(60) Provisional application No. 63/438,152, filed on Jan. 10, 2023.

(51) Int. Cl. G16H 40/20; G06F 3/0482; G06Q 10/0631

(52) U.S. Cl. CPC G16H 40/20; G06F 3/0482; G06Q 10/06315

(57) ABSTRACT

Disclosed are systems and methods that provide a novel framework for the real-time management and control of electronic/digital and/or physical activities performed in, around and/or in relation to a healthcare facility. The disclosed framework provides an interactive user interface (UI) that displays, as immersive and interactive interface cards, real-time digital data and content that corresponds to the digital and/or physical activities. The interactive interface cards are selectable, and configured with portal capabilities for the discovery of additional information, the creation of new forms of data, and the interaction with other users, entities, departments and other interactive capabilities in/around the facility. The disclosed framework enables a fully interactive, personalized and dynamic management platform for controlling operations of a facility while maintaining management control of those operations.

### CROSS-REFERENCE TO RELATED APPLICATIONS

This application claims the benefit of priority from U.S. Provisional Application No. 63/438,152, filed Jan. 10, 2023, whereby the contents of which are incorporated herein by reference.

### FIELD OF THE DISCLOSURE

The present disclosure is generally related to digital data management, and more particularly, to digital management and control of electronic activity data related to real-time activities and information associated with at least one location.

### BACKGROUND

Currently, many healthcare facilities, inclusive of hospitals, physician offices and urgent care centers, among others, are tasked with the undertaking of managing the operations in and around the facility while maintaining a reasonable amount of available resources. For example, during the COVID-19 pandemic, hospitals struggled to identify available resources to adequately address patient's needs. Thus, the lack of understanding of available beds, medical supplies and physician availability can lead to a decrease in the quality of care.

### SUMMARY OF THE DISCLOSURE

According to some embodiments, the disclosed systems and methods provide a novel computerized framework that provides functionality for addressing such shortcomings, among others, by providing a digital management platform that enables the real-time control (which includes near real-time control which may be subject to network and other communications delays and the like) of a facility, inclusive of the manner supplies, resources and people are available and/or where they are scheduled. Indeed, as evident from the disclosed systems and methods, the disclosed framework enables real-time communication between entities (e.g., companies, third parties, people, staff, physicians, suppliers, vendors, and the like) that can facilitate dynamic decision making processes, which can be performed in a timely, resource efficient manner. This can lead to the reduction of resource drain, an increased experience of care for patients, and improved overall efficiency in the manner a facility operates.

According to some embodiments, as discussed herein, the disclosed framework can enable the identification of available supplies, resources, people and space, for example, that can be computationally analyzed in a manner that enables patient care to be accurately, efficiently and properly assigned and performed. For example, rather than simply monitoring whether supplies are running low or are out of stock, as in conventional mechanisms, the disclosed framework can leverage modern technology to manage and run a healthcare facility. For example, as discussed herein, according to some embodiments, the disclosed framework can dynamically and automatically monitor real-time activity data of a facility, and the entities associated therewith (e.g., vendors, for example), and determine current needs and/or predicted needs of the facility. These needs can be leveraged into actionable tasks automatically performed by the disclosed framework, which can prevent circumstances where patient care becomes less viable, not available or below a dedicated standard of care expected/required from such facilities.

In some embodiments, the disclosed framework can present a platform user interface (UI) that can be dynamically configured for a particular entity, service, person, location, facility type, information type, task type, and the like, or some combination thereof. In some embodiments, the UI can enable interaction with and the creation of new forms of data and/or analyses. In some embodiments, the UI can enable communication with other users and/or entities (e.g., different departments, customers or vendors, for example).

According to some embodiments, the UI can present such data and/or created data/analysis as interactive interface cards (e.g., interface object (IO) cards) that are fully interactive, and can trigger actionable steps, as discussed herein. The interactive interface cards can be modified, re-positioned, shared, added, removed and the like, from the UI. According to some embodiments, the interface cards can present any type of known or to be known forms of data and/or content, including, but not limited to, text, images, video, multimedia, graphs, statistics, augmented reality (AR), virtual reality (VR), extended reality (XR), and the like. In some embodiments, the UI can enable interaction with the cards as a display similar to the virtual display of the metaverse.

As such, according to some embodiments as discussed herein, the disclosed are systems and methods provide a novel framework for that enables the real-time management of digital and/or physical activities performed in, around and/or in relation to a healthcare or any other facility. The disclosed framework provides an interactive UI that displays, as immersive and interactive interface cards, real-time digital data and content that corresponds to the digital and/or physical activities. The interactive interface cards are selectable, and configured with portal capabilities for the discovery of additional information, the creation of new forms of data, and the interaction with other users, entities, departments and other interactive capabilities in/around the facility. The disclosed framework enables a fully interactive, personalized and dynamic management platform for controlling operations of a facility while maintaining management control of those operations.

According to some embodiments, a method is disclosed for real-time digital management and display in association with operations of users, services and/or activities of and/or associated with a location. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for a novel and improved framework for real-time digital management and display in association with operations of users, services and/or activities of and/or associated with a location.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary management environment according to some embodiments of the present disclosure;

FIG. 4 illustrates an exemplary digital management environment according to some embodiments of the present disclosure;

FIGS. 5A and 5B illustrate an exemplary digital management environment according to some embodiments of the present disclosure;

FIGS. 6A, 6B and 6C illustrate an exemplary digital management environment according to some embodiments of the present disclosure;

FIGS. 7A and 7B illustrate an exemplary digital management environment according to some embodiments of the present disclosure;

FIG. 8 illustrates an exemplary digital management environment according to some embodiments of the present disclosure;

FIG. 9 illustrates an exemplary digital management environment according to some embodiments of the present disclosure;

FIG. 10 illustrates an exemplary data flow according to some embodiments of the present disclosure;

FIG. 11 illustrates an exemplary data flow according to some embodiments of the present disclosure;

FIG. 12 depicts an exemplary implementation of a cloud computing architecture according to some embodiments of the present disclosure;

FIG. 13 depicts an exemplary implementation of a cloud computing architecture according to some embodiments of the present disclosure; and

FIG. 14 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments will now be described in greater detail with reference to the figures. According to some embodiments as discussed herein, the disclosed systems and methods provide a novel framework that enables the real-time management of digital and/or physical activities performed in, around and/or in relation to a healthcare or any other facility. The disclosed framework provides a comprehensive networked platform via an interactive UI that displays, as immersive and interactive interface cards, real-time digital data and content that corresponds to the digital and/or physical activities. The interactive interface cards are selectable, and configured with portal capabilities for the discovery of additional information, the creation of new forms of data, and the interaction with other users, entities, departments and other interactive capabilities in/around the facility. The disclosed framework enables a fully interactive, personalized and dynamic management platform for controlling operations of a facility while maintaining management control of those operations.

As discussed herein, in some embodiments, the disclosed framework provides a user facing communication tool that enables the identification and creation of data that can be leveraged to provide care to patients. For example, the framework's UI can be patient facing, in that the patient user can view and interaction with information related to, but not limited to, their medical chart, prescribed medications and/or treatments, analytics related to medication/treatment, physician/staff information (e.g., background, demographics and/or ratings of physicians, for example), and the like, or some combination thereof. Accordingly, in another example, the framework's UI can be facility facing (or entity facing), thereby enabling viewership and/or interaction with information related to, patient, staff, vendors, operations and activities of a facility, as discussed herein.

In some embodiments, the interactive interface cards of the UI, and/or the UI as a whole, can provide interfaces that enable the creation and/or viewing of medical related data. For example, such data can include, but is not related to, educational quizzes, surveys, recommended content (e.g., videos, images, simulations, and the like), games, notifications, message boards, chat interfaces, and the like, or some combination thereof, as discussed below in more detail.

In some embodiments, the data can also or alternatively provide information related to resources of a facility (or multiple facilities). For example, the data can correspond to, but is not limited to, medical equipment, supplies, medication, available physicians, nurses, staff, specialists, and the like, or some combination thereof. In some embodiments, the data, for example, can indicate current quantities of available medical supplies (e.g., medications, bandages, and the like, for example). In some embodiments, the data can also provide available medical professionals and their schedules. For example, a neurologist is currently in the operating room (OR), but becomes available at a later time X.

Thus, in some embodiments, the resources of a facility can correspond to any type of care provided to a patient, which can include, but is not limited to, medical supplies, physicians, nurses, staff, parking, beds, rooms, waiting room, wheel chairs, and the like. In some embodiments, the resource data may be associated with a facility and/or a vendor or provider to the facility. For example, if a vendor is responsible for providing medication Y, then the data can indicate a next scheduled shipment to the facility and/or other information related to the contractual agreement for the vendor to provide such data. Thus, for example, if the data indicates that medication Y is running low, then the data can provide capabilities to display when the medication will be refilled by a particular vendor. In some embodiments, the UI and/or interactive interface cards can enable capabilities for the automated purchase of additional supplies on a one time or periodic basis. Prompts to users can be provided along with these automated purchase capabilities to approve the purchases as desired.

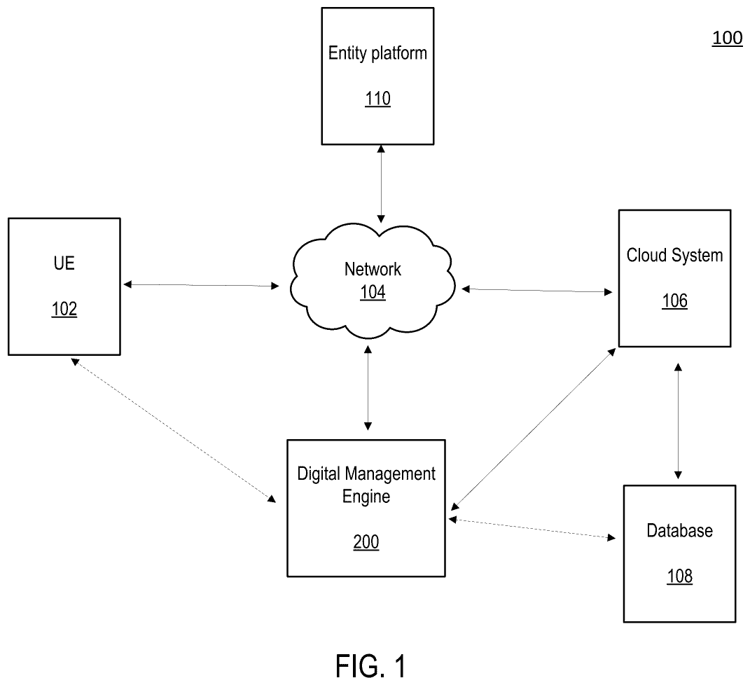

According to some embodiments, as discussed below, the data associated with the displayed content in the UI and interface cards can be hosted and/or stored in an operational database (ODB) and/or clinical database (CDB). Thus, for example, database 108 of FIG. 1, as discussed below can be a database(s) that provides ODB and CDB capabilities and functionality.

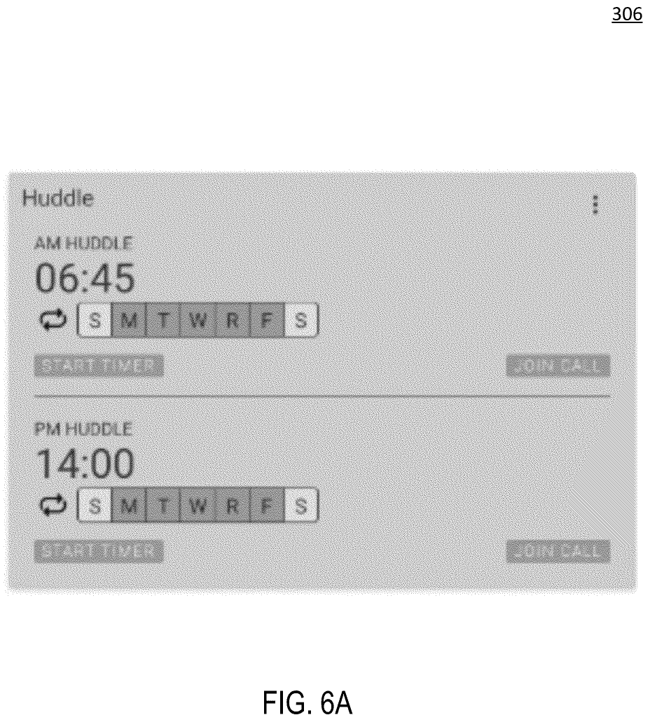

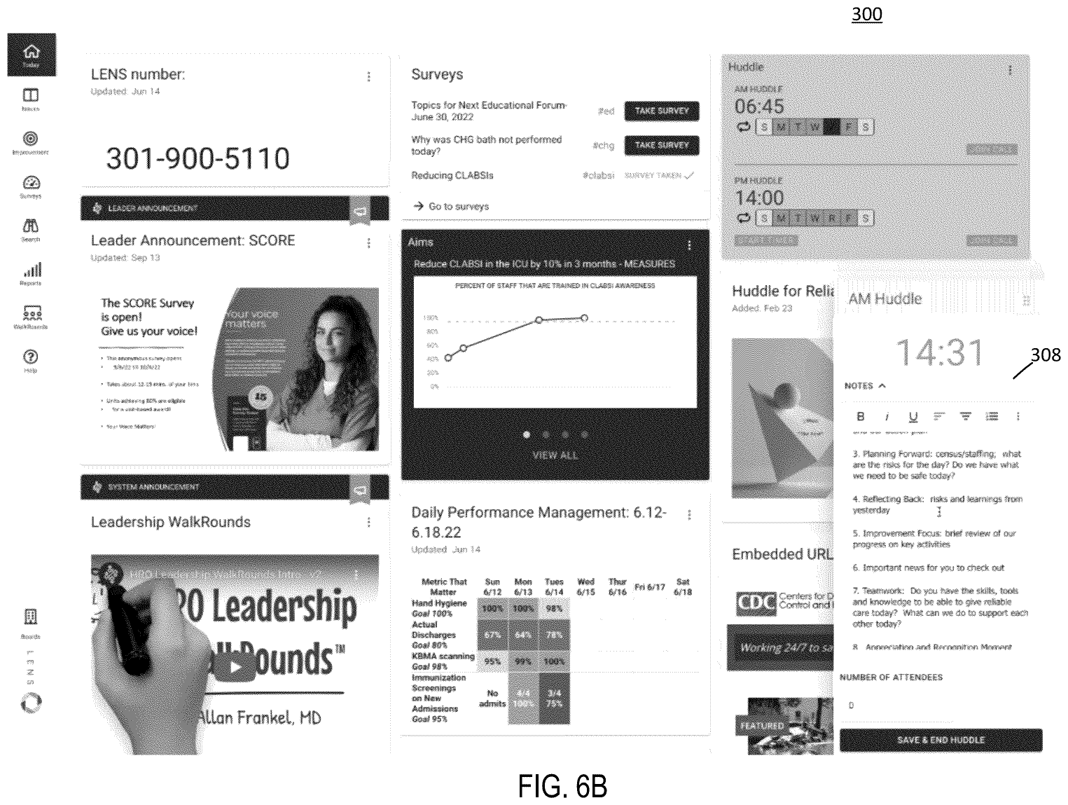

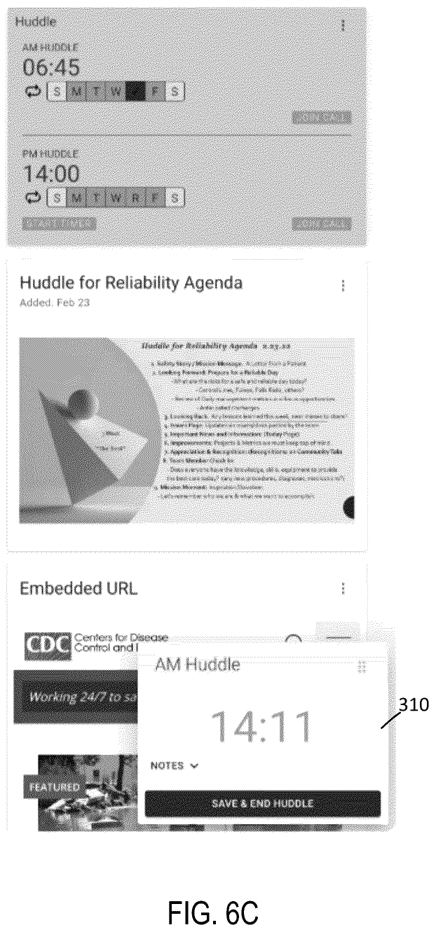

According to some embodiments, as discussed below, the disclosed framework, via the UI, can enable “huddle” capabilities. For example, as depicted in FIGS. 6A-6C, as discussed in more detail below, users can engage in real-time and/or scheduled electronic meetings via a provided interface within the UI. The interface can enable multiple users that are either registered and/or invited to the huddle meeting to interact in a real-time, collaborative environment. In some embodiments, the huddle can enable text interaction, video interaction, sharing of documents to work collaboratively, and the like, or some combination thereof. In some embodiments, the huddle capabilities can be configured to operate in connection with a user's calendar thereby enable scheduling and/or activation via calendaring capabilities.

In some embodiments, the interface cards can be associated with and/or provide capabilities for data, programs, widgets, application program interfaces (APIs), networks, portals, and/or any other type of content, software, network and/or functionally capable of being interacted with via a displayed UI on a device (inclusive of, but not limited to, a mobile device, networked device, peripheral device and/or virtual machine).

According to some embodiments, the framework/platform may further be configured with functionality to compile simulations and/or renderings that provide guidance and/or learning experiences for teaching users how to operate software, perform tasks, and/or interact with the UI. For example, the UI may be configured to display, in an interactive manner, a display window that renders a multimedia rendering for performing a task (e.g., performing a medical procedure), where the rendering includes a computationally determined, step-by-step procedure for the task based on preoperative, post-operative and/or operative information derived from medical imaging (e.g., computed tomography (CT), magnetic resonance imaging (MRI), compressed sensing (CS) and the like).

By way of a non-limiting example, an MRI image can be captured, and analyzed, whereby specifics of a patient's condition can be determined. For example, a preoperative image of a patient's knee can reveal that a revision anterior cruciate ligament (ACL) surgery is required (based on a prior ACL surgery not being successful). This image can be computationally analyzed via the framework implementing any type of known or to be known computational analysis, machine learning (ML) and/or artificial intelligence (AI) algorithm, classifier and/or technique, including, but not limited to, computer vision, neural network analysis, and the like. In some embodiments, for example, the framework can then identify information related to remedying particular issues with similar types of conditions of the patient (e.g., a search of a database of known ACL revision procedures). In some embodiments, the framework can then compile a step-by-step procedure as an interactive simulation, that can then be rendered by a requesting user (e.g., a physician performing the ACL revision procedure). In some embodiments, such rendering can be activated and/or launched from a dedicated interface card strategically and automatically displayed within the UI for that particular user.

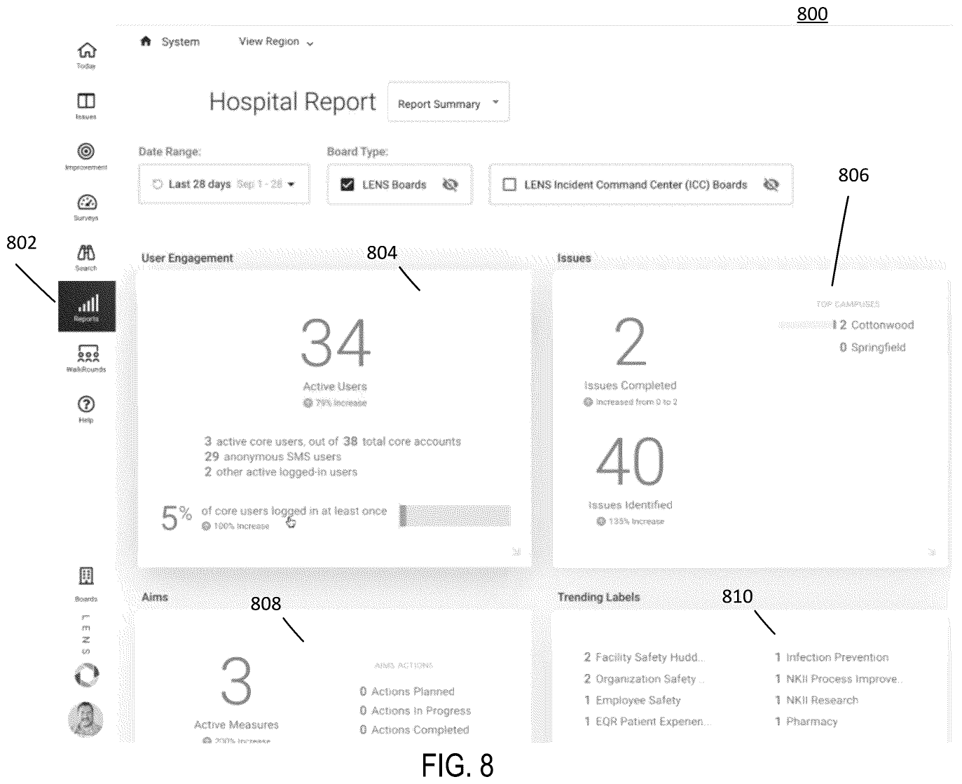

In some embodiments, as discussed below, the UI can also display windows and/or electronic pages/documents related to hospital reports. As discussed below in relation to FIG. 8, the reports, which can be customized per a criteria (e.g., time, date, location, user, patient, and the like), can indicate, but is not limited to, which users are active, goals for procedures and/or management of the facility, issues at/around the facility, and trending issues, among others. In some embodiments, the reports can provide benchmarks that can be utilized to determine an overall efficiency and safety of the facility.

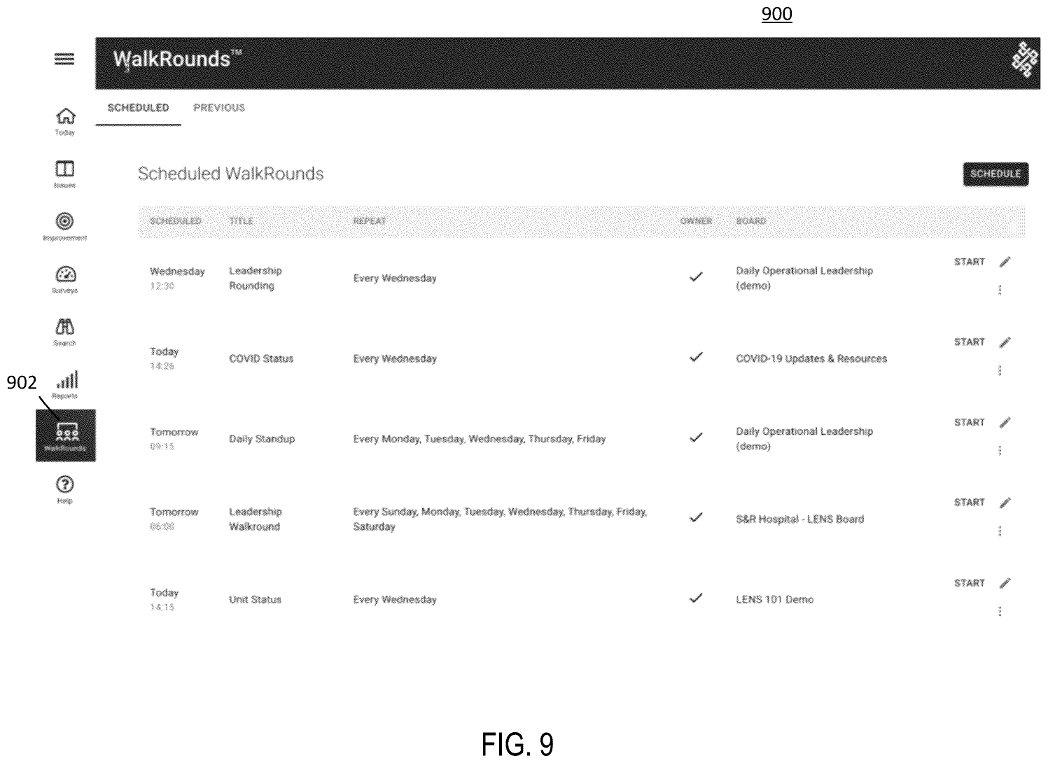

In some embodiments, as discussed below at least in relation to FIG. 9, the UI can also display a window and/or an electronic page related to “WalkRounds®” (“WR”) pages. WalkRounds® pages, as discussed in more detail below, enable safety rounds to be performed for a facility, which can include, but are not limited to, equipment checks, patient checks, staff checks, physician checks and/or any other type of intervention or analysis of an operation at the facility. In some embodiments, the WalkRounds® page can be computationally determined based on patient and/or medical professional schedules and/or activities to provide an organized, sequential and interactive schedule that can be displayed for which assigned uses can be alerted and/or engage in tasks.

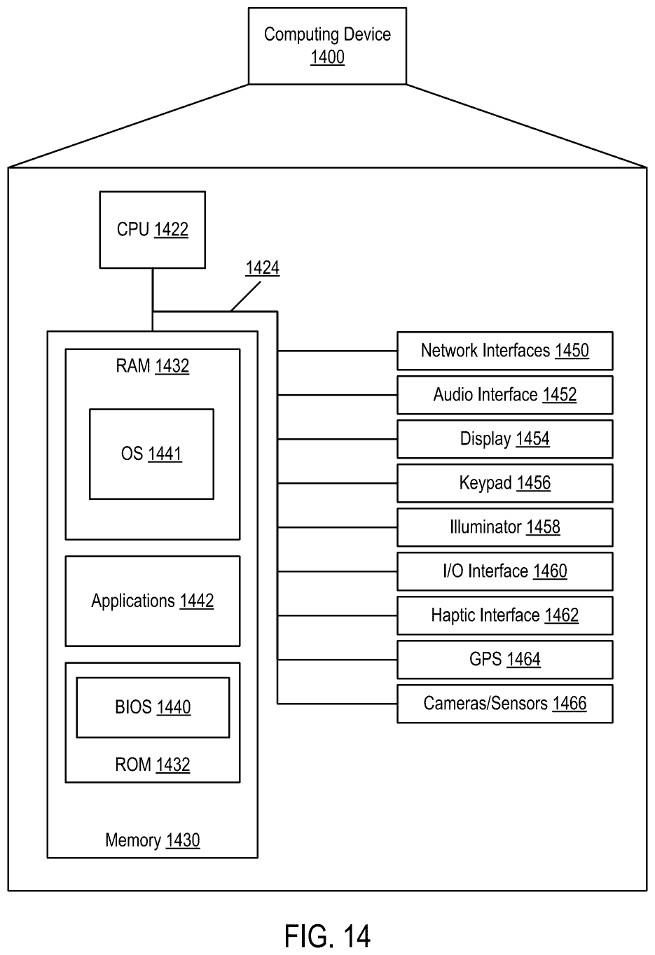

With reference to FIG. 1, system 100 is depicted which includes UE 102 (e.g., a client device, as mentioned above and discussed below in relation to FIG. 14), entity platform 110, network 104, cloud system 106, database 108 and digital management engine 200. It should be understood that while system 100 is depicted as including such components, it should not be construed as limiting, as one of ordinary skill in the art would readily understand that varying numbers of UEs, entity platforms (e.g., servers, for example), cloud systems, databases and networks can be utilized; however, for purposes of explanation, system 100 is discussed in relation to the example depiction in FIG. 1.

According to some embodiments, UE 102 can be any type of device, such as, but not limited to, a mobile phone, tablet, laptop, sensor, Internet of Things (IoT) device, autonomous machine, and any other device equipped with a cellular or wireless or wired transceiver. In some embodiments, UE 102 can be associated with a user (e.g., a medical professional, officers of a facility (e.g., chief medical officer (CMO), for example), third party vendor, and the like), entity, business, company, vendor, network, portal, provider, and the like. In some embodiments, UE 102 can be associated with a user associated with entity platform 110.

According to some embodiments, an entity platform 110 can correspond to a server(s) associated with an entity. It should be understood that platform 110 can contain a single or multiple servers, and can be any type of server, including, but not limited to, a banking server, authentication server, search server, email server, social networking server, SMS server, IM server, MMS server, exchange server, enterprise server, and the like. Thus, the entities associated with platform 110 can be any type of entity that is, but is not limited to, associated with a medical facility, associated with providing healthcare, associated with providing products, instruments, medications and/or equipment to medical and/or healthcare locations, and the like, or some combination thereof. Thus, an entity associated with entity platform 110 can be any type of company, business, user, network, firm, account, application, agency, government, wallet, vendor, medical professional, physician, nurse, and the like; and can be connected to network 104 and utilize the functionality provided by engine 200, as hosted by cloud system 106, as discussed herein.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service provider, network provider and/or medical provider from where services and/or applications may be accessed, sourced or executed from. For example, system 106 can represent the cloud-based architecture associated with at least one medical facility location (e.g., a hospital or urgent care facility) which has associated network resources hosted on the internet or private network (e.g., network 104), which enables (via engine 200) the digital data management discussed herein.

In some embodiments, for example, system 106 can provide UE 102 and/or a user associated with entity platform 110 with a programming interface that enables a search for data, whereby the search identifies a set of data that can be leveraged for performing the data management discussed herein (e.g., determining available goods/services/products available for a facility, for example, as discussed herein). Therefore, in some embodiments, system 106 can host and/or provide a network resource(s) that enables users access to engine 200's capabilities—for example, the network resource can be a web page, web site, portal, application, and the like, or some combination thereof.

In some embodiments, cloud system 106 may include a server(s) and/or a database of information which is accessible over network 104. In some embodiments, a database 108 of cloud system 106 may store a dataset of data and metadata associated with local and/or network information related to a user(s) of UE 102 and the UE 102, entity platform 110, and the services and applications provided by cloud system 106 and/or digital management engine 200.

In some embodiments, for example, cloud system 106 can provide a private/proprietary management platform, whereby engine 200, discussed infra, corresponds to the novel functionality system 106 enables, hosts and provides to a network 104 and other platforms operating thereon. Accordingly, according to at least some embodiments, system 106 may be configured to receive, handle, process, manage, monitor, settle and/or decline transaction requests to/from users and/or entities (e.g., entity platform 110).

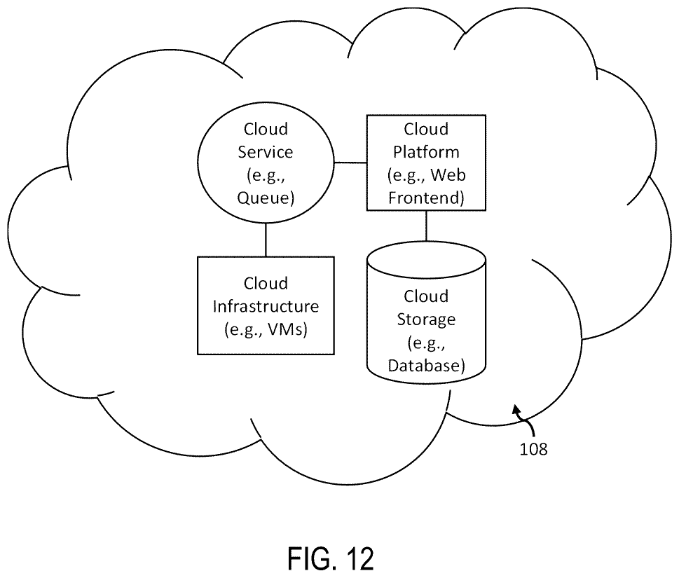

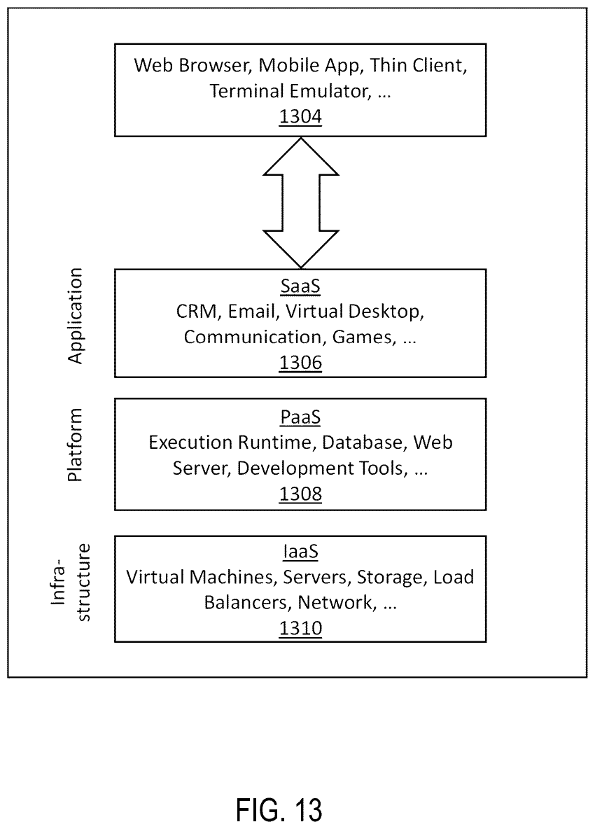

Turning to FIGS. 12 and 13, in some embodiments, the exemplary computer-based systems/platforms, the exemplary computer-based devices, and/or the exemplary computer-based components of the present disclosure may be specifically configured to operate in a cloud computing/architecture 108 such as, but not limiting to: infrastructure a service (IaaS) 810, platform as a service (PaaS) 808, and/or software as a service (SaaS) 806 using a web browser, mobile app, thin client, terminal emulator or other endpoint 804. FIGS. 12 and 13 illustrate schematics of non-limiting implementations of the cloud computing/architecture(s) in which the exemplary computer-based systems for administrative customizations and control of network-hosted and/or blockchain-related APIs via a workflow service (and/or microservice) of a blockchain environment of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). As discussed above, in some embodiments, database 108 can provide ODB and CDB capabilities.

According to some embodiments, database 108 may correspond to a distributed ledger of a distributed network. In some embodiments, the distributed network may include a plurality of distributed network nodes, where each distributed network node includes and/or corresponds to a computing device associated with at least one entity (e.g., the entity associated with cloud system 106, for example, discussed supra). In some embodiments, each distributed network node may include at least one distributed network data store configured to store distributed network-based data objects for the at least one entity. For example, database 108 may correspond to a blockchain, where the distributed network-based data objects can include, but are not limited to, account information, medical information, entity identifying information, wallet information, device information, network information, credentials, security information, permissions, identifiers, smart contracts, transaction history, and the like, or any other type of known or to be known data/metadata related to an entity's and/or user's information, structure, business and/or legal demographics, inter alia.

In some embodiments, a blockchain may include one or more private and/or private-permissioned cryptographically-protected, distributed databased such as, without limitation, a blockchain (distributed ledger technology), Ethereum (Ethereum Foundation, Zug, Switzerland), and/or other similar distributed data management technologies. For example, as utilized herein, the distributed database(s), such as distributed ledgers ensure the integrity of data by generating a digital chain of data blocks linked together by cryptographic hashes of the data records in the data blocks. For example, a cryptographic hash of at least a portion of data records within a first block, and, in some cases, combined with a portion of data records in previous blocks is used to generate the block address for a new digital identity block succeeding the first block. As an update to the data records stored in the one or more data blocks, a new data block is generated containing respective updated data records and linked to a preceding block with an address based upon a cryptographic hash of at least a portion of the data records in the preceding block. In other words, the linked blocks form a blockchain that inherently includes a traceable sequence of addresses that may be used to track the updates to the data records contained therein. The linked blocks (or blockchain) may be distributed among multiple network nodes within a computer network such that each node may maintain a copy of the blockchain. Malicious network nodes attempting to compromise the integrity of the database must recreate and redistribute the blockchain faster than the honest network nodes, which, in most cases, is computationally infeasible. In other words, data integrity is guaranteed by the virtue of multiple network nodes in a network having a copy of the same blockchain. In some embodiments, as utilized herein, a central trust authority for sensor data management may not be needed to vouch for the integrity of the distributed database hosted by multiple nodes in the network.

In some embodiments, exemplary distributed blockchain-type ledger implementations of the present disclosure with associated devices may be configured to affect transactions involving Bitcoins and other cryptocurrencies into one another and also into (or between) so-called FIAT money or FIAT currency, and vice versa.

In some embodiments, the exemplary distributed blockchain-type ledger implementations of the present disclosure with associated devices are configured to utilize smart contracts that are computer processes that facilitate, verify and/or enforce negotiation and/or performance of one or more particular activities among users/parties. For example, an exemplary smart contract may be configured to be partially or fully self-executing and/or self-enforcing. In some embodiments, the exemplary inventive asset-tokenized distributed blockchain-type ledger implementations of the present disclosure may utilize smart contract architecture that may be implemented by replicated asset registries and contract execution using cryptographic hash chains and Byzantine fault tolerant replication. For example, each node in a peer-to-peer network or blockchain distributed network may act as a title registry and escrow, thereby executing changes of ownership and implementing sets of predetermined rules that govern transactions on the network. For example, each node may also check the work of other nodes and in some cases, as noted above, function as miners or validators.

Digital management engine 200, as discussed above and further below in more detail, can include components for the disclosed functionality. According to some embodiments, digital management engine 200 may be a special purpose machine or processor, and can be hosted by a device on network 104, within cloud system 106 and/or on UE 102. In some embodiments, engine 200 may be hosted by a server and/or set of servers associated with cloud system 106.

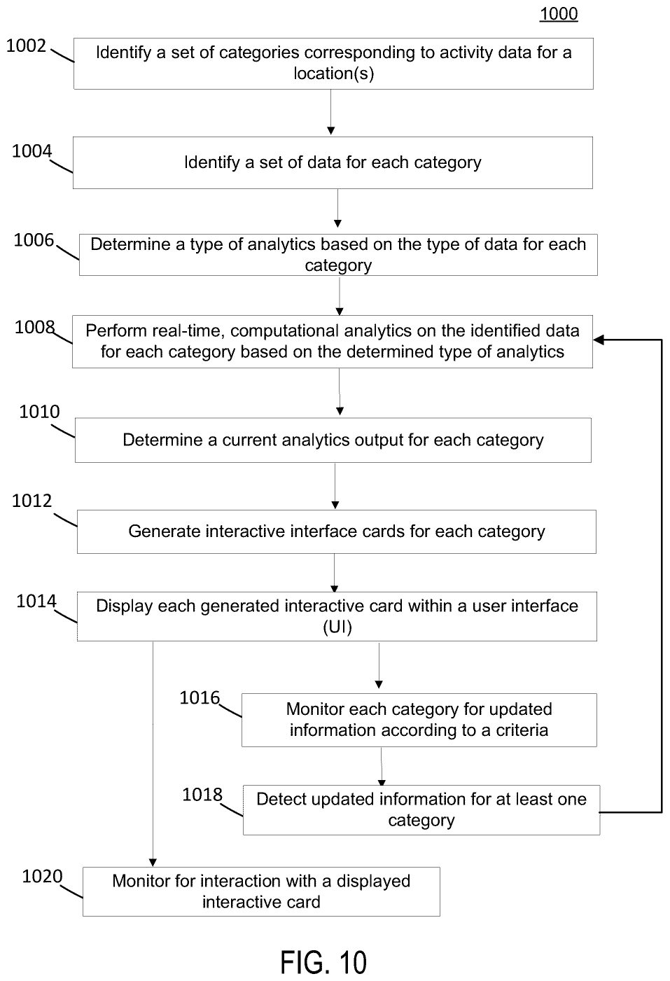

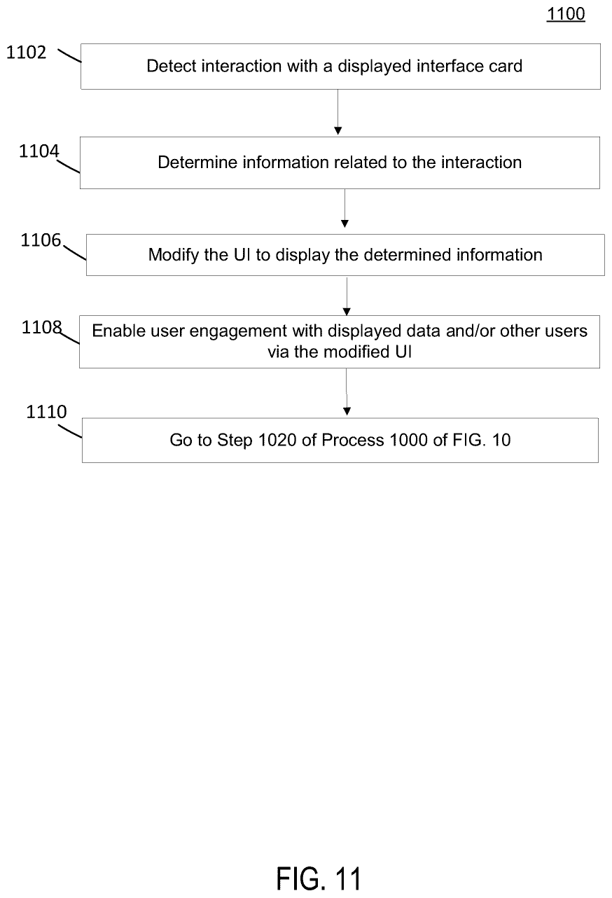

According to some embodiments, as discussed in more detail below, digital management engine 200 may be configured to implement and/or control a plurality of services and/or microservices, where each of the plurality of services/microservices are configured to execute a plurality of workflows associated with performing the disclosed digital data management. Non-limiting embodiments of such workflows are provided below in relation to at least FIGS. 10-11.

According to some embodiments, as discussed above, digital management engine 200 may function as an application provided by cloud system 106. In some embodiments, engine 200 may function as an application installed on a server(s), network location and/or other type of network resource associated with system 106. In some embodiments, engine 200 may function as application installed and/or executing on UE 102 and/or entity platform 110. In some embodiments, such application may be a web-based application accessed by UE 102 and/or servers associated with entity platform 110 over network 104 from cloud system 106 (e.g., as indicated by the connection between network 104 and engine 200). In some embodiments, engine 200 may be configured and/or installed as an augmenting script, program or application (e.g., a plug-in or extension) to another application or program provided by cloud system 106 and/or executing on UE 102 and/or entity platform 110.

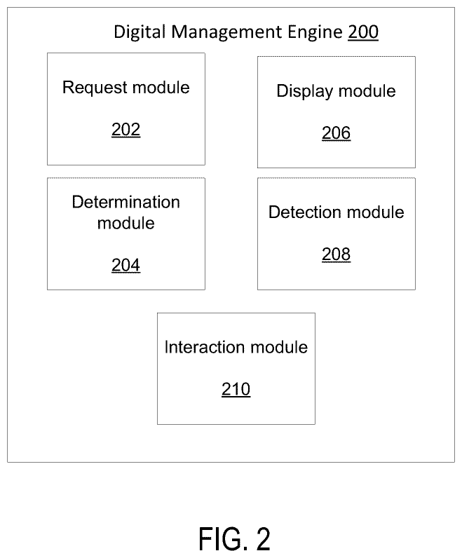

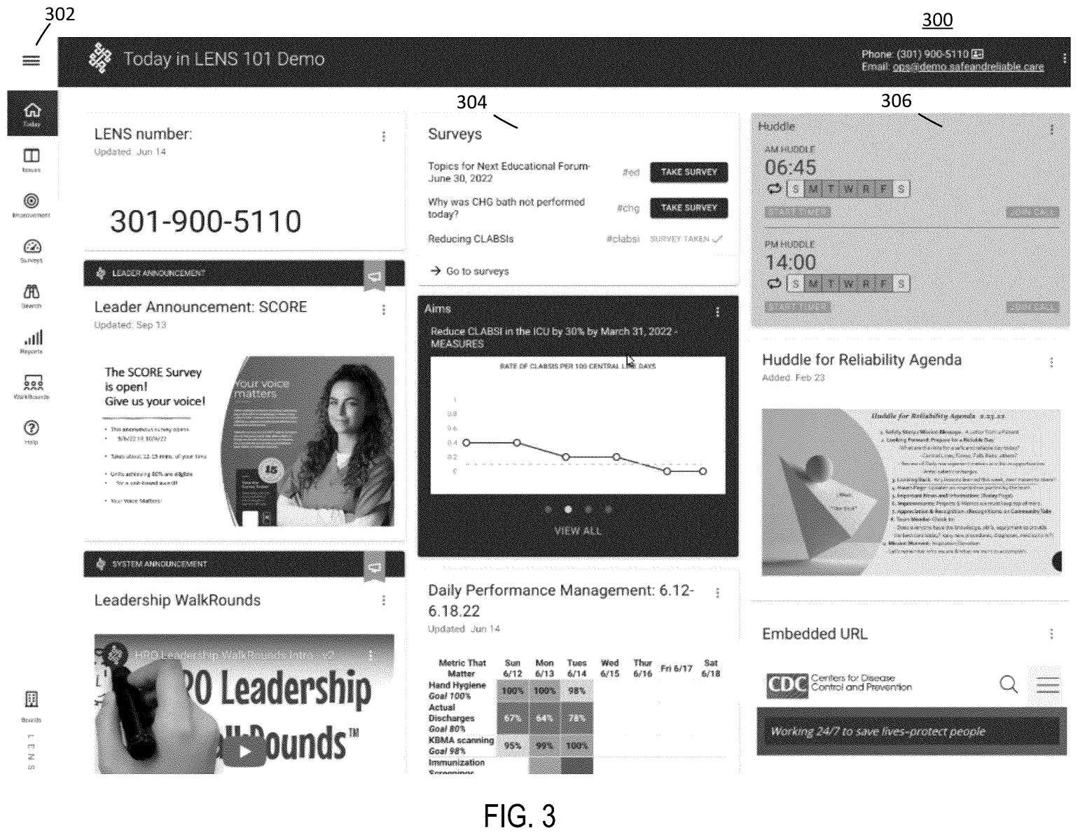

As illustrated in FIG. 2, according to some embodiments, digital management engine 200 includes request module 202, determination module 204, display module 206, detection module 208 and interaction module 210. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below in relation to FIGS. 3-11, inter alia.

Turning to FIG. 3, provided is an exemplary management environment according to some embodiments of the present disclosure. FIG. 3 depicts an example user interface (UI) 300, which provides an electronic document (or page) for display on a display of a device (e.g., UE 102, for example). As discussed herein, in some embodiments, UI 300 displays a menu bar 302 and interactive interface cards 304 and 306.

It should be understood that for purposes of this discussion, cards 304 and 306 will be discussed as example cards; however, it should not be construed as limiting, as other types of cards, data and/or capabilities via the cards and UI 300 are embodied in the disclosed embodiments, as discussed above and in more detail below.

According to some embodiments, UI 300 can be configured as a personalized, customizable and/or automatically determined network environment where a user(s) may view and/or interact with activity and/or operational data related to at least one facility. In some embodiments, UI 300 can be compiled and presented for a single user, and in some embodiments, UI 300 can be for a set of users (e.g., nurses working in a particular department, for example).

In some embodiments, UI 300 can be compiled and displayed based on computational analysis of contextual information about a user. In some embodiments, UI 300 can be based on selected interactive interface cards and/or categories of information by a user. In some embodiments, UI 300 can be a combination of both embodiments.

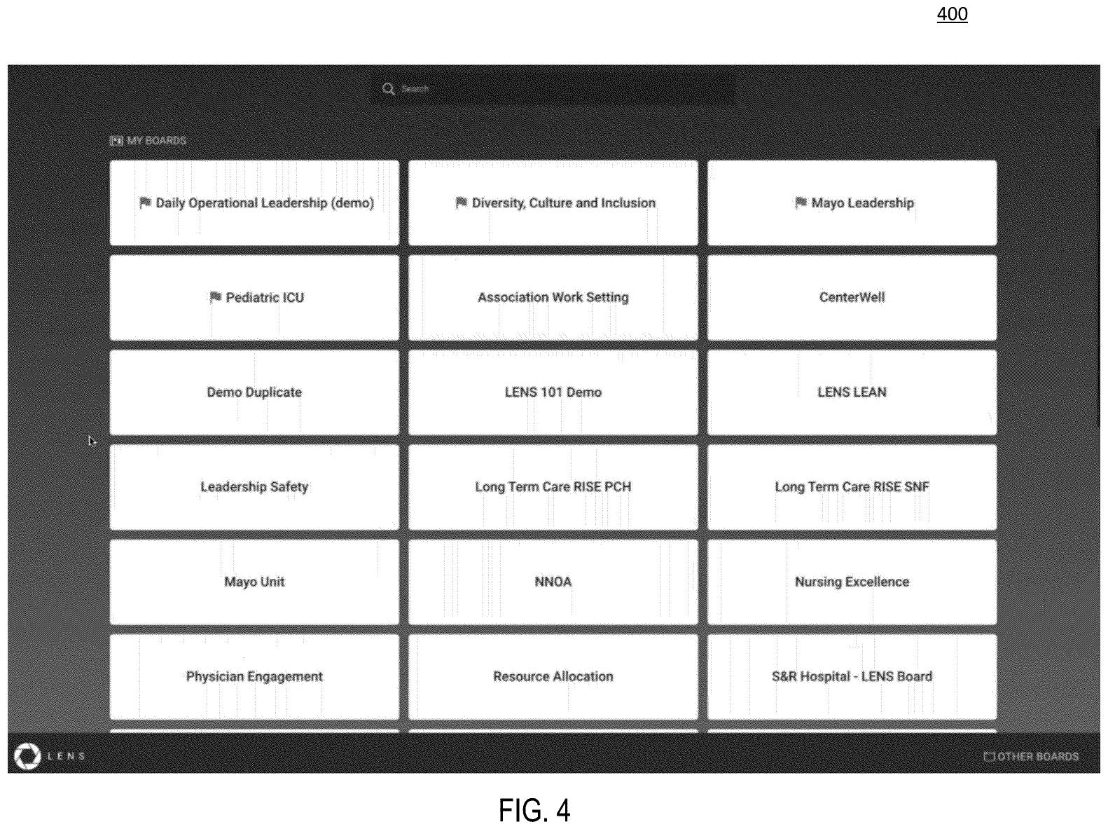

For example, as depicted in FIG. 4, UI 400 provides a non-limiting embodiment of an electronic page that can be displayed to a user. UI 400 depicts a set of cards that represent categories of information which correspond to particular types of data related to operations of a facility. For example, a card labeled “Pediatric ICU” can correspond to a set of analytical data (e.g., for a particular time period) for the pediatric intensive care unit (ICU) of a hospital. As discussed herein, a user can view UI 400 and select each card that they desire to display within UI 300. In some embodiments, the types and/or quantity of cards may be dependent on the type of user. For example, more sensitive information related to patients and/or operations of the hospital may be reserved for users with specific credentials.

Further discussion of the compilation of the UI 300 and the mechanisms for populating UI 300 with cards and/or interacting with such cards is discussed in more detail below in relation to FIGS. 10-11.

Turning back to FIG. 3, according to some embodiments, interactive interface cards (also referred to as “interface cards” or “cards”, interchangeably) 304 and 306 are real-time interactive interface objects that are configured to automatically display and/or enable interaction with fetched, pushed, determined and/or hosted information. Each card 304 and 306 (and other cards depicted and/or capable of being depicted within UI 300) provide their own personalized interactive, networked environment for the exploration of data and the creation of a content consumption experience for a dedicated set of users.

In some embodiments, cards 304 and 306 can be specific to a type or category of information, and may be customized for the viewing user (e.g., the user for which UI 300 is generated). In some embodiments, for example, the cards displayed on UI 300 can be selected, positioned and/or manipulated based on a criteria related to, but not limited to, an identity of a user, input by a user, security clearance of a user, location of a user, type of device of the user, and the like, or some combination thereof.

In some embodiments, the data/content depicted in each card may be based on, but not limited to, a category of data/content, type of data/content, location, time period, contract, agreement, type of user, and the like, or some combination thereof.

For example, card 304 can provide surveys. In some embodiments, the types of surveys can be based on a user type and/or types of data being leveraged for the surveys. For example, if the survey is for the CMO, then the survey may include more sensitive information related to the operations of the hospital. However, if the survey is for a nurse, the survey may include data/questions relating to patient care for the wing and/or department the nurse works.

Thus, in some embodiments, the UI 300 and the cards provided therein, can be automatically displayed, updated and/or modified based on, but not limited to, a type of user, type of data, time period, type of facility, location of the facility, regulations for the facility, and the like, or some combination thereof.

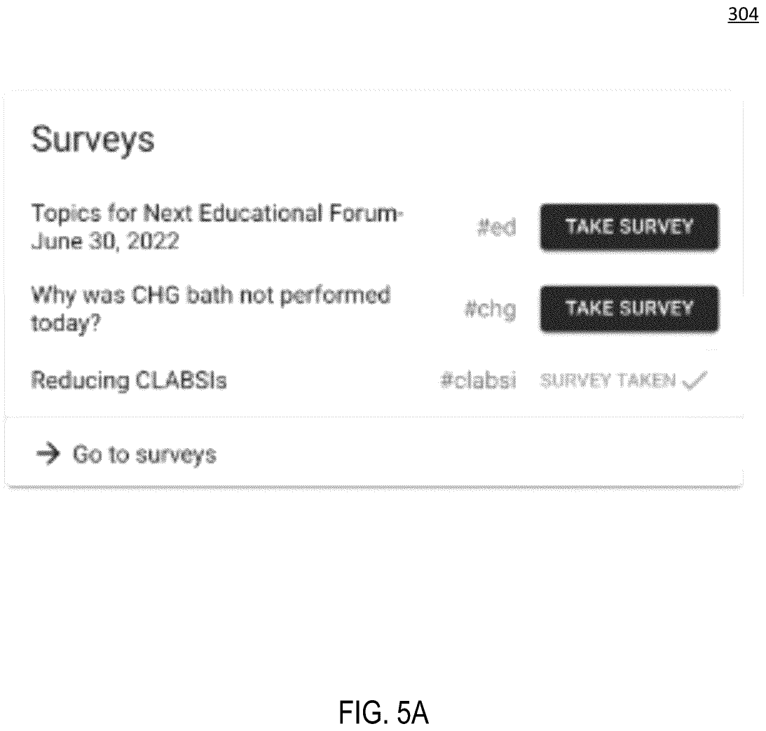

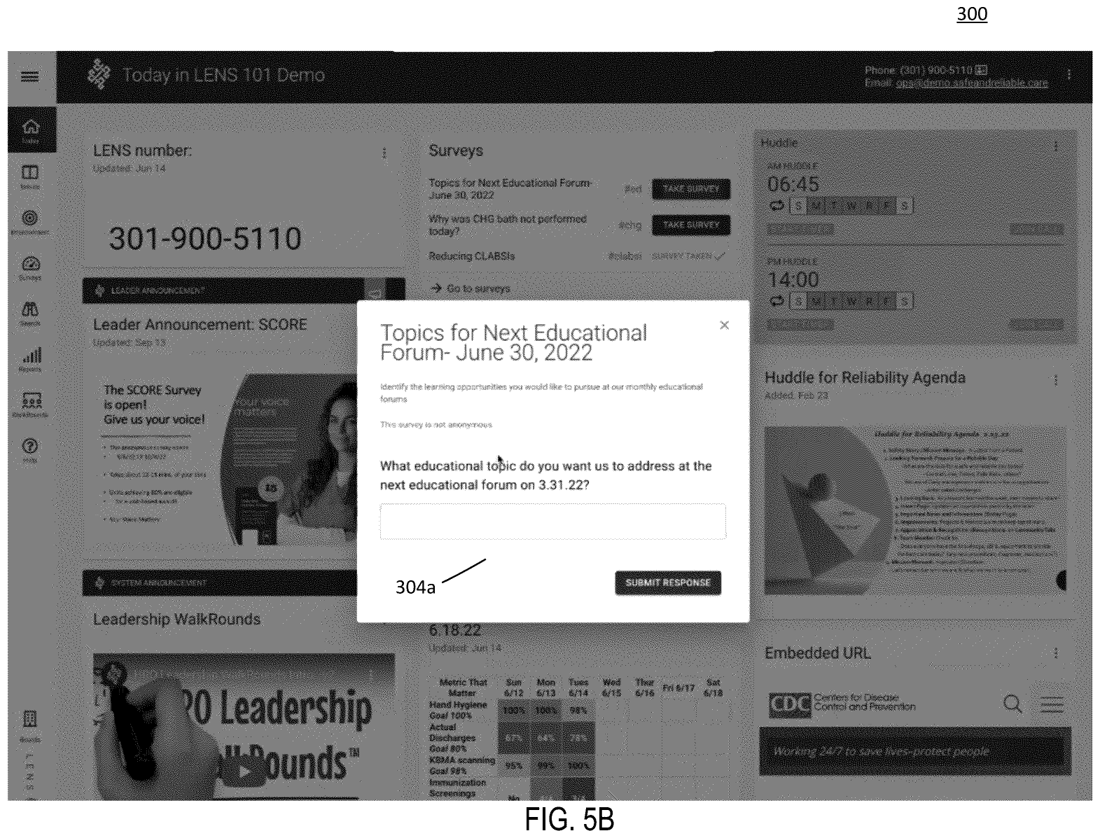

Turning to FIG. 5A, depicted is card 304, which is an example survey card. In some embodiments, survey card 304 can be configured to display a plurality of IOs related to particular surveys, where, in some embodiments, an IO can be for a specific survey. In some embodiments, the IOs can be interactive and provide information related to, but not limited to, a name or title of the survey, a category, type and/or tag associated with the survey (e.g., a hashtag, for example), and a button for engaging with the survey, as depicted in FIG. 5A.

According to some embodiments, a survey can be a set of interactive questions and/or requested inputs that are compiled to gather information, which can be related to other activities and/or operations of a facility and/or related to particular users, equipment, vendors and/or entities. For example, a survey can be set up by a unit manager, whereby users selected to complete the surveys can be identified based on the types of questions and/or requested information from the survey. For example, in some embodiments, upon compilation of a survey, the disclosed framework can analyze the survey and determine the type of information being request, then automatically identify the users that are capable of providing such information. In some embodiments, this identification can be based on contextual analysis of the user's information and/or behavior at/around the facility. Thus, in some embodiments, the survey can be auto-populated in the survey card 304 for that particular user(s). In some embodiments, creating entity of the survey can select which users are to receive the survey.

Thus, according to some embodiments, as depicted in FIG. 5A, survey card 304 can be personalized per user, and can indicate whether a survey has been completed, if it is in progress and/or whether an expiration date for the survey is approaching. In some embodiments, survey card 304 can enable interaction per survey via the “Take Survey” button, or can enable a user to be presented with a screen that modifies the UI 300 to present a listing of all surveys assigned to a user—for example, via selection of the “Go to surveys” button.

FIG. 5B depicts an example of a selected survey from survey card 304 of FIG. 5A. For example, interface screen 304a is depicted with displays a survey that enables a user to enter information related to the requested information, and subsequently submit the response (which can be sent to the surveyor and/or other parties, and stored in a database).

Turning to FIG. 6A, depicted is huddle card 306. As discussed above, huddle card 306 enables users to engage in real-time and/or scheduled electronic meetings via a provided interface within UI. Huddle card 306, and its subsequent and/or related interfaces 308 and 310 (as depicted in FIGS. 6B and 6C, respectively) can enable multiple users, that are either registered and/or invited to the huddle meeting, to interact in a real-time, collaborative environment. In some embodiments, a huddle can enable text interaction, video interaction, sharing of documents to work collaboratively, and the like, or some combination thereof. In some embodiments, huddle capabilities can be configured to operate in connection with a user's calendar thereby enable scheduling and/or activation via calendaring capabilities.

Therefore, as depicted in FIG. 6A, huddle card 306 provides an example of two scheduled huddles for a user—for example, a huddle beginning at 06:45 and another at 14:00. Each scheduled huddle indicates a time of day and a day of the week for the scheduled huddle. For example, the 06:45 huddle is scheduled for Monday through Friday. Huddle card 306 enables the user to begin a timer for a scheduled huddle, and/or join a call (or initiate a call) with the other parties in the huddle.

In some embodiments, each huddle can be set/scheduled for a period of time. In some embodiments, a huddle may be scheduled for a predetermined period of time (e.g., 15 minutes); whereby a timer can be presented as a count-down for remaining time of a huddle. In some embodiments, despite a predetermined or preset time, the huddle time can be extended, which can impact how UI 300 is displayed and/or the huddle window 308 is displayed, as depicted in FIG. 6B

FIG. 6B illustrates an example of an on-going huddle via window 308, which can be overlaid the display of UI 300. Window 308, in some embodiments, can be embedded in UI 300, displayed in another UI and/or appended as a toolbar/sidebar to UI 300. Window 308 provides electronic capabilities for users assigned to and/or engaging in the huddle to communicate in real-time. For example, window 308 indicates a number of attendees, an agenda for the huddle meeting and/or an input area for user's to engage. For example, window 308 can provide group chat functionality for all huddle attendees. In some embodiments, window 308 can be collapsible (and expandable), as depicted in window 310 of FIG. 6C.

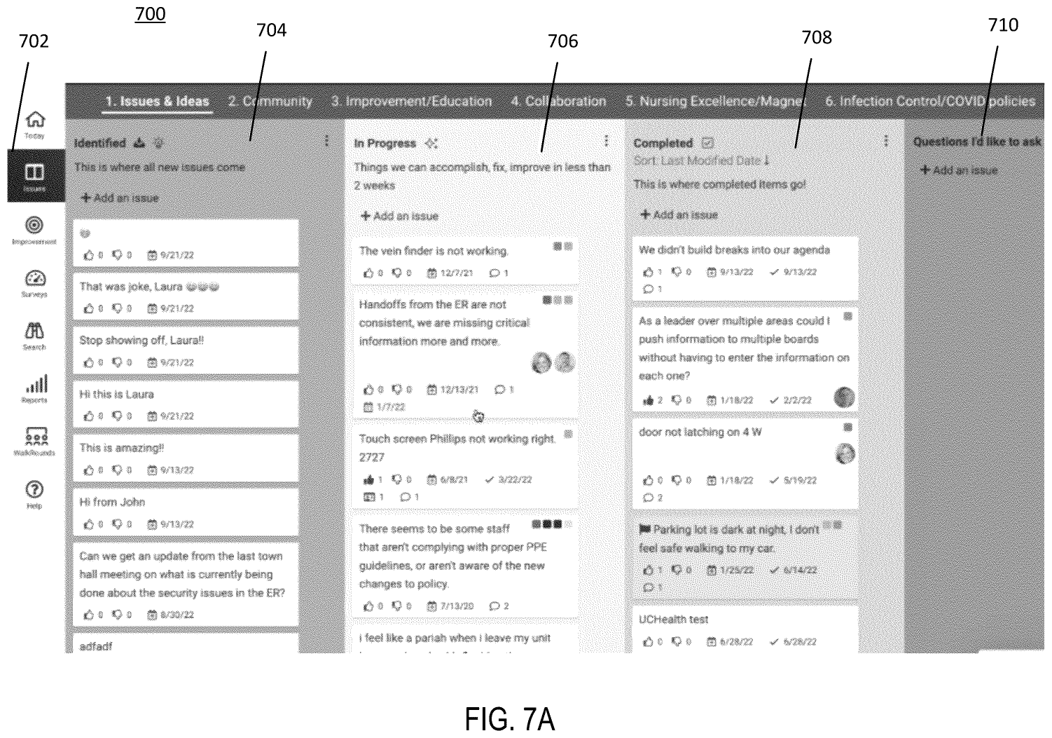

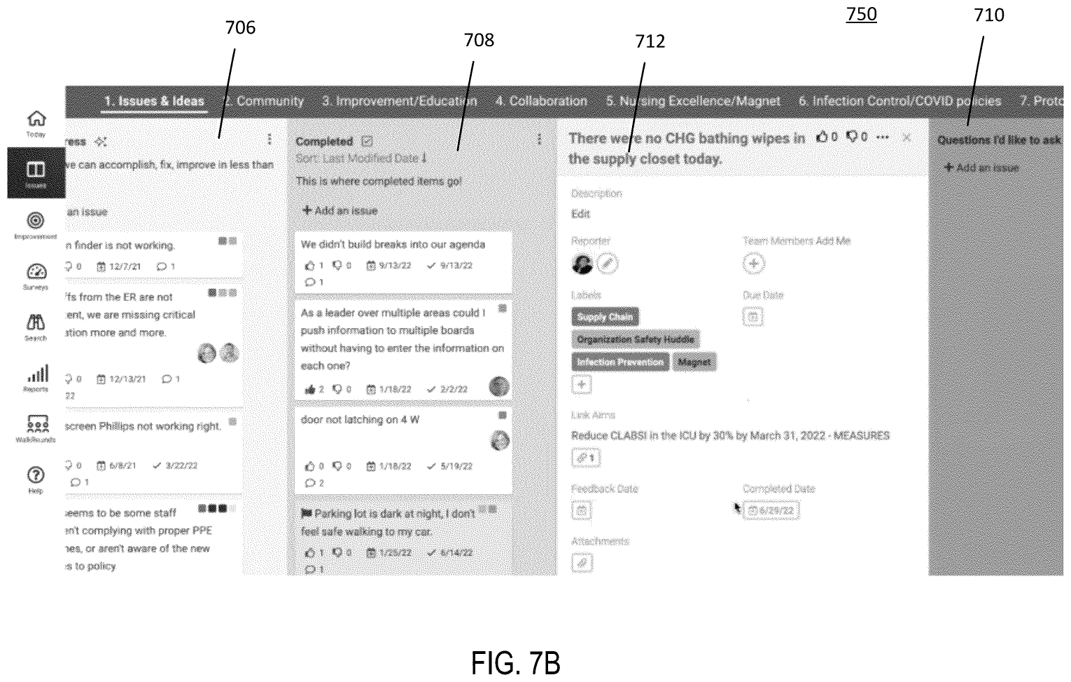

Turning to FIG. 7A, depicted is UI 700, which corresponds to an online environment provided by the disclosed framework for users to engage with activities and/or operations of a facility. In some embodiments, as depicted in FIG. 7A, UI 700 can be displayed upon interaction with element 702 from menu bar 302. Thus, in some embodiments, the elements in menu bar 302 enable the toggling of display screens associated with each element depicted in menu bar 302. In some embodiments, UI 700 (and UIs 800 and 900, as discussed below, and UI 400, discussed supra) may be displayable upon a user selecting a corresponding interface card from UI 300.

According to some embodiments, UI 700 includes sections 704, 706, 708 and 710. Each section can display information within them as interface objects, which can enable users to, but no limited to, like, share, chat, engage and/or communicate with each other and/or the status of the topic of the interface object (and/or any other type of feedback). In some embodiments, each interface object can further display a date, which can correspond to which section 704-708 it is being displayed in (e.g., a date for when it populated in a particular section).

In some embodiments, section 704 can correspond to a display section of UI 700 where issues at the facility can be listed. In some embodiments, the listed issues can be automatically populated and displayed in section 704. In some embodiments, users can send issue requests to the framework, whereby they can be analyzed and determined as an issue to be displayed. In some embodiments, the ordering of the issues can be based on, but not limited to, a type of issue, time of issue, time of issue request/alert, type of user providing alert to issue, priority of issue, and the like, or some combination thereof. In some embodiments, issues can be grouped according to categories of issues (e.g., issues with lighting can be grouped for maintenance).

In some embodiments, section 706 can correspond to a display section of UI 700 that indicates which issues have been identified and are currently in progress of being addressed. In some embodiments, therefore, the interface objects for each issue presented in section 704 can be moved to section 706 (and subsequently to section 708, as discussed below). In some embodiments, the movement of interface objects between sections can be, but is not limited to, drag and drop operations, selections, automatic determinations that an issue's state has changed (e.g., a user indicates they are handling the issue, therefore it moves to section 706), and the like, or some combination thereof. Therefore, this evidences the capabilities of UI 700 whereby UI 700 and its sections 704-708 can be automatically modified to display, interchange and modify interface objects corresponding to particular issues. For example, an interface object displayed in section 706 can be removed from section 704 and re-populated in section 706.

In some embodiments, the interface objects displayed in section 706 can be expanded to allow and/or display notes to be provided related to the progress on the particular issue. In some embodiments, additional sub-windows or pop-up interfaces may be capable of being displayed to provide additional or supplemental information related to the issue and/or progress. In some embodiments, such functionality (e.g., expansion and/or supplemental display) may be capable within section 704.

In some embodiments, section 708 displays a window/UI portion for which issues have been completed (or fixed). The interface objects for the issues “in progress” from section 706 can be moved to section 708 in a similar manners as the interface objects from sections 704 and 706, discussed supra. In some embodiments, the interface objects in section 708 can also be expandable and/or initiated to display supplemental windows, as discussed above.

In some embodiments, section 710 displays an area where questions can be added. The questions can be input in relation to sections 704-708. In some embodiments, upon an input being provided via section 710, UI 700 can be modified to create an interface object for that specific input and have it displayed within section 704, 706 and/or 708, whereby its display may correspond to whether it is a new issue, is in progress and/or is related to a completed issue. Thus, in some embodiments, the input in section 710 may result in modification of an existing interface card (e.g., the input relates to the progress of addressing an existing issue; therefore, an interface object in section 706 is modified to include the updated input).

FIG. 7B provides a modified version of UI 700, whereby UI 750 displays section 712 that can be displayed upon interaction with a specific interface object within sections 704-708. As discussed above, interface objects within sections 704-708 are interactive and expandable; therefore in some embodiments, additional/supplemental information about an issue (e.g., whether it is new, in progress or completed) can be requested to be displayed, whereby section 712 can be presented within UI 750.

According to some embodiments, section 712 can display, but is not limited to, tags (or labels) of the issue, a topic/title of the issue (e.g., what was input in section 710), links to network resources related to the issue, attachments, dates of the issue, its progress and completion, identifiers of users related to the issue (e.g., who reported the issue), team members assigned to completing the issue, a due date for the completion of the issue, feedback dates and capabilities for editing the issue (e.g., should the issue get worse, it can be edited to expand upon its detail), and the like, or some combination thereof.

Turning to FIG. 8, depicted is UI 800, which provides an electronic page depicting a hospital report. According to some embodiments, the hospital report (or facility report, as it can pertain to any type of facility or medical unit) can be based on, but not limited to, a time period, current time, date range, location, unit/department, set of users, set of issues, goals/aims, and the like, or some combination thereof. In some embodiments, the report can be compiled continuous periodically (e.g., every 2 hours, for example) and/or according to received requests from authorized users.

In some embodiments, as depicted in FIG. 8, UI 800 can be displayed upon interaction with element 802 from menu bar 302. In some embodiments, UI 800 may be displayable upon a user selecting a corresponding interface card from UI 300.

According to some embodiments, as mentioned above, UI 800 depicts the hospital report, whereby each section of the report can be interactive and/or independently updateable (e.g., capable of being updated without a need for other sections). In some embodiments, the information displayed within UI can be created, requested, determined, customized and/or modified according to a preset and/or provided criteria, which can be, but is not limited to, time, date, location, user, patient, and the like, or some combination thereof. In some embodiments, the information displayed within the report depicted in UI 800 can indicate, but is not limited to, which users are active 804, issues at/around the facility 806, goals for procedures and/or management of the facility 808, and trending issues 810, among others, and the like, and/or some combination thereof. Thus, each portion and/or type of information can be displayed as an interactive section 804-810 of UI 800. It should be understood that any type of data related to a facility and/or its operation, as discussed above (see, e.g., FIGS. 3-4, for example) can be displayed as part of the hospital report.

As mentioned above, in some embodiments, the reports can provide benchmarks that can be utilized to determine an overall efficiency and safety of the facility. For example, if the issues detected number (e.g., within section 806) displayed in UI 800 is at or above a threshold, then this can provide an alert to an administrator. In some embodiments, the benchmarks (e.g., thresholds) can be preset, in accordance with a type of data, and/or dynamically determined according to the criteria discussed above.

In FIG. 9, UI 900 is depicted, which corresponds to the electronic page for WalkRounds®. In some embodiments, as depicted in FIG. 9, UI 900 can be displayed upon interaction with element 902 from menu bar 302. In some embodiments, UI 900 may be displayable upon a user selecting a corresponding interface card from UI 300.

WalkRounds® UI 900 can enable safety rounds to be performed for a facility, which can include, but are not limited to, equipment checks, patient checks, staff checks, physician checks and/or any other type of intervention or analysis of an operation at the facility. In some embodiments, UI 900 can be computationally determined based on patient and/or medical professional schedules and/or activities to provide an organized, sequential and interactive schedule that can be displayed for which assigned uses can be alerted and/or engage in tasks.

By way of example, a WR for a user can be set by a managing user of the facility. In another non-limiting example, a WR for a user can be determined based on analysis of the user's calendar and/or tracking of the user's device (e.g., via location-tracking) which can compile a schedule of behavior of the user. This can be leveraged via the ML/AI analysis discussed above, for example, to determine a schedule of tasks for the user to perform safety checks. The determined schedule of tasks can be displayed in UI 900, whereby each task can be interactive and/or provide indications of their status, location (both in the physical facility and networked facility (e.g., cards displayed within UI 300, for example), and the like, or some combination thereof. In some embodiments, a user can create a new WR and assign it to themselves and/or other users via the “schedule” button depicted in UI 900.

FIG. 10 provides Process 1000 which details non-limiting example embodiments for disclosed digital data management. According to some embodiments, the disclosed framework (via engine 200) provides a time-based (e.g., daily or shift-based, for example) management system via an interactive, automatically and dynamically modifiable UI that displays, as immersive and interactive interface cards, real-time digital data and content that corresponds to the digital and/or physical activities. The interactive interface cards are selectable, and configured with portal capabilities for the discovery of additional information, the creation of new forms of data, and the interaction with other users, entities, departments and other interactive capabilities in/around the facility. As discussed above and in detail herein, disclosed framework enables a fully interactive, personalized and dynamic management platform for controlling operations of a facility while maintaining management control of those operations.

According to some embodiments, Steps 1002-1004 of Process 1000 can be performed by request module 202 of digital management engine 200; Steps 1006-1010 can be performed by determination module 204; Steps 1012-1014 can be performed by display module 206; and Steps 1016-1020 can be performed by detection module 208.

According to some embodiments, Process 1000 begins with Step 1002 where engine 200 receives a request to display UI 300. According to some embodiments, the request can be provided by a user, set of users, department, facility, administrator, provider, entity, vendor, company, and the like, or some combination thereof. In some embodiments, the request can be automatically triggered based on a detected criteria that corresponds to, but is not limited to, a time, date, activity and the like. For example, engine 200 may receive a request each time a physician shows up for a shift, thereby enabling them to gauge the current status of their department, staff, patients and equipment.

In some embodiments, the request of Step 1002 can include information related to, but not limited to, a set of categories corresponding to activity data for a location. In some embodiments, as discussed above, a location can correspond to a medical facility. In some embodiments, the location may correspond to a sup-part of the facility (e.g., department). In some embodiments, the location may correspond to a geographical region (e.g., a plurality of facilities) that treat a segment of a particular population.

In some embodiments, the activity data, as discussed above, can correspond to, but is not limited to, a time period, real-time data, specific users, specific equipment, specific operations, and the like, or some combination thereof. Thus, in effect, in some embodiments, the request in Step 1002 may correspond to a request for a specified set of interactive interface cards, as discussed above at least in relation to FIGS. 3-4, inter alia.

In Step 1004, engine 200 can identify a set of data for each category. In some embodiments, the set of data can be specific to a location and/or time period. In some embodiments, the set of data can be retrieved from database 108, as discussed above. Accordingly, in some embodiments, the identified set of data can provide an indication of activity data for categorized operations/activities occurring at the location according to a particular time (or time period/range).

In Step 1006, engine 200 can determine a type of analytics to be perform on the identified type of data. That is, in some embodiments, engine 200 can determine whether to perform certain computations based on the certain types of data being requested. In some embodiments, the analytics can correspond to particular software programs, APIs, executable add-ons and/or third party applications that enable data to be computationally analyzed for the creation of new forms of data that provides insights into the status of a facility.

According to some embodiments, such computational analysis discussed herein can involve any type of known or to be know AI/ML and/or large language model(s). According to some embodiments, engine 200 can execute a specific trained AI/ML model, a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 200 may leverage an LLM, whether known or to be known. As discussed herein, an LLM is a type of AI system designed to understand and generate human-like text based on the input it receives. The LLM can implement technology that involves deep learning, training data and natural language processing (NLP). Large language models are built using deep learning techniques, specifically using a type of neural network called a transformer. These networks have many layers and millions or even billions of parameters. LLMs can be trained on vast amounts of text data from the internet, books, articles, and other sources to learn grammar, facts, and reasoning abilities. The training data helps them understand context and language patterns. LLMs can use NLP techniques to process and understand text. This includes tasks like tokenization, part-of-speech tagging, and named entity recognition.

LLMs can include functionality related to, but not limited to, text generation, language translation, text summarization, question answering, conversational AI, text classification, language understanding, content generation, and the like. Accordingly, LLMs can generate, comprehend, analyze and output human-like outputs (e.g., text, speech, audio, video, and the like) based on a given input, prompt or context. Accordingly, LLMs, which can be characterized as transformer-based LLMs, involve deep learning architectures that utilizes self-attention mechanisms and massive-scale pre-training on input data to achieve NLP understanding and generation. Such current and to-be-developed models can aid AI systems in handling human language and human interactions therefrom.

In some embodiments, engine 200 may be configured to utilize one or more AI/ML techniques selected from, but not limited to, computer vision, feature vector analysis, decision trees, boosting, support-vector machines, neural networks, nearest neighbor algorithms, Naive Bayes, bagging, random forests, logistic regression, and the like.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
e. apply the trained model to process the newly received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

For example, if the request in Step 1002 is from a radiologist at a hospital, and is for MRI imagery of a particular patient and tracked behavioral movements of the nursing staff for a particular time period, then engine 200 analyze the request and the data retrieved for each request and determine that the MRI's can be analyzed via computer vision analysis and the behaviors can be tracked via a trained deep belief network to produce predicted routes/rounds (e.g., WR) for each nurse. Accordingly, the markers and/or “progress” for the nursing staff can be collected, which can correspond to, but not be limited to, assigned tasks, steps taken, equipment used, equipment discarded, equipment requested, patient progress, location within the hospital, and the like, or some combination thereof. Moreover, such collected data can correspond to historical data which can provide similar information from all and/or similarly related tasks.

In another non-limiting example, a hospital Chief Execute Officer (CEO), via the disclosed systems and methods (e.g., engine 200 and the functionality provided by the GUIs of FIGS. 3, 4, 6A-9, inter alia) can effectively track the progress of a nurse handling tasks involving the use and expenditure of hospital equipment. According the some embodiments, a hospital CEO can implement comprehensive monitoring and assessment functionality enabled via engine 200 and the corresponding UIs provided via the instant disclosure. According to some embodiments, the CEO may establish key performance indicators (KPIs) related to the nurse's responsibilities, such as, but not limited to, equipment utilization efficiency, maintenance records, adherence to protocols, and the like. Regular reports detailing equipment usage, associated costs, and any incidents, for example, can be (and may be required to be) submitted by the nurse or relevant department. Additionally, the CEO might employ a digital tracking system at the hospital that records real-time data on equipment usage, ensuring transparency and accuracy (e.g., via sensor UE 102, discussed supra).

In some embodiments, to assess the quality of the nurse's performance, the CEO may conduct periodic reviews, considering factors such as, but not limited to, patient outcomes, adherence to safety protocols, and feedback from both patients and colleagues. Such data can be collected via such sensors, and/or provided via feedback from the nurse, colleagues, patients, the CEO, and the like, or some combination thereof. In some embodiments, regular performance evaluations can be scheduled to discuss achievements, challenges, and areas for improvement. In some embodiments, the CEO can utilize technology to monitor the nurse's adherence to best practices, utilizing electronic health records and task management systems to ensure proper documentation and follow-through. For example, in some embodiments, computer vision of the nurses movements captured on image frames from hospital cameras can be utilized to determine adherence to hospital and ethical protocols of the health profession.

In some embodiments, financial aspects, such as, but not limited to, budget adherence and cost-effectiveness, can be closely monitored by integrating expenditure reports into the assessment process. In some embodiments, financial reviews related to the nurse's activities can provide insights into resource management and identify opportunities for optimization. For example, cost of equipment used, and whether such usage was required can be determined via engine 200 executing such AI/ML and/or LLM technologies, discussed supra. In some embodiments, the CEO may also communicate, via electronic messaging according to each progress reporting, professional development messaging for the nurse, ensuring that they stay abreast of the latest technologies and best practices in equipment usage and patient care.

While the systems and methods described herein have been described in healthcare environments as non-limiting examples, in some embodiments, such systems and methods are also useful in industrial, education, agriculture, information technology, financial services, mining, and software environments as well as many others. As just one example, an industrial application can include some or all of the functionalities listed herein to enable better communication, maintenance and management of manufacturing personnel, processes, inputs and outputs.

Accordingly, the disclosed technology discussed herein can enable a hospital CEO to track and quantify a nurse's performance in handling tasks involving equipment by implementing a multifaceted approach that includes setting clear KPIs, utilizing digital tracking systems, conducting regular performance evaluations and closely monitoring financial aspects. Such comprehensive strategy ensures effective oversight, promotes accountability and facilitates continuous improvement in both the nurse's performance and the hospital's overall quality of care.

In Step 1008, engine 200 can perform computational analytics on the identified data for each category based on the determined type of analytics from Step 1006. In some embodiments, the execution of the analytics can be performed in real-time (e.g., upon the request based on real-time collected data) and/or can be based on a collected set of data (e.g., periodically performed according to predetermined intervals). In some embodiments, engine 200 can perform at least a portion of the determined analytics in parallel, and in some embodiments, can perform at least a portion of the determined analytics in a sequential order (e.g., at least those analytics determined to be based on other analytics). Such comprehensive analytics can be performed via any of the AI/ML and/or LLM techniques discussed supra.

In Step 1010, engine 200 can determine, based on the execution of the computational analytics (from Step 1008), a current analytics output for each category. That is, in some embodiments, Step 1010 can provide or produce the output of the computational analysis of the analysis performed in Step 1008. For example, the computer vision-based analysis of the MRI images can be compiled as a set of renderings for subsequent display.

In Step 1012, interactive interface cards for each category can be generated by engine 200. The generation/creation and display of the categorical information can be performed in a similar manner as discussed above at least in relation to FIGS. 3-6A. In some embodiments, the interface cards can be created and customized to provide a preview of the data, such that upon interaction with the card, a new or modified UI may be displayed that enables further display of the information collected and determined via Process 1000. Examples of this are discussed above, and can be found at least in relation to FIGS. 3-9, supra.

In Step 1014, engine 200 facilitates the display of the generated interface cards within a UI. According to some embodiments, this is discussed, for example, at least in relation to FIG. 3, supra.

In some embodiments, Process 1000 can proceed to Step 1016, whereby engine 200 can monitor the network associated with the location and devices connected thereto for updated information for each category according to one or more criteria. In some embodiments, the monitoring can be continuous or according to a predetermined time period, event detection or criteria associated therewith. In some embodiments, as discussed above, the criteria may be, but is not limited to, a time, type of activity, user, and the like. For example, continuing with the above example, if a nurse missed a check-in with a patient, this may trigger an update of the data for a particular category corresponding to the nursing staff's rounds.

Thus, in Step 1018, in some embodiments, engine 200 may detect updated information for a particular category (e.g., at least one category associated with a displayed interface card within the UI) based on the monitoring of Step 1016. In some embodiments, upon such detection, engine 200 can recursively proceed back to Step 1008 for performing computational analysis based on the updated information. Accordingly, in some embodiments, the detection of the updated information can cause an interactive interface card to be dynamically updated, while it is displayed, without user interaction or a subsequent request.

According to some embodiments, Process 1000 may proceed from Step 1014 to Step 1020. In some embodiments, engine 200 can proceed from Step 1014 to Step 1016 and from Step 1014 to Step 1020, whereby Steps 1014 and 10120 can be performed simultaneously (or substantially simultaneously). Therefore, in some embodiments, engine 200 can effectively perform both steps, and they need not be performed at the same time.

In some embodiments, Step 1020 can involve engine 200 monitoring for interaction with a displayed interactive card. As discussed above, such monitoring can be time-based, user-based and/or activity-based. Upon detection of the interaction, engine 200 can perform Process 1100 of FIG. 11, discussed below. An example of such interaction detection can be found at least in relation to the disclosure of FIGS. 5A-9, discussed above.

Turning to FIG. 11, Process 1100 is shown which details non-limiting example embodiments for management and control of the UI that enables digital data management, as discussed herein. According to some embodiments, Steps 1102 and 1110 of Process 1100 can be performed by detection module 208; Step 1104 can be performed by determination module 204; Step 1106 can be performed by display module 206; and Step 1108 can be performed by interaction module 210.

According to some embodiments, Process 1100 begins with Step 1102 where engine 200 detects interaction with a displayed interactive interface card (e.g., based on the monitoring from Step 1020 of Process 1000). In some embodiments, such interaction can be, but is not limited to, an input, detected event, and the like, that corresponds to the interactive display/elements of the interface card, as discussed above.

In Step 1104, engine 200 can analyze the interaction, and determine information related to the interaction and/or the interface card. For example, engine 200 can determine what type of information is being requested, which type of interface object and/or window is to be displayed, determine if there is updated information related to the content of the interface card being interacted with, whether the user has access to the requested data, and the like, or some combination thereof. In some embodiments, Step 1104 can be performed by engine 200 via any type of known or to be know computational analysis ML/AI classifier, algorithm or technique, as discussed above.

In Step 1106, engine 200 can modify the display of the UI to display the determined information. Non-limiting example embodiments of this modification can be found from the disclosure above at least in relation to FIGS. 3-9.

In Step 1108, engine 200 can enable user engagement with the displayed data and/or other users via the modified UI. For example, as discussed above at least in relation to FIGS. 6A-6C, a huddle card can be interacted with, whereby a window can be displayed that enables engagement with other assigned/participating users of the huddle. In another non-limiting example embodiments, as discussed above in relation to FIGS. 7A-7B, an interactive card can be selected which triggers UI 700/750 to be displayed, which enables the capabilities discussed above for UI 700/750.

According to some embodiments, as a result of the user engagement enabled via Step 1108, engine 200 can proceed to Step 1110, which can trigger/execute Step 1020 of Process 1000 for monitoring for interaction with the now modified UI. Thus, in some embodiments, processing by engine 200 can revert back to Step 1020 of Process 1000 to monitor for interaction with the UI and the information/content being displayed therein.

FIG. 14 is a schematic diagram illustrating a client device showing an example embodiment of a client device that may be used within the present disclosure. Client device 1400 may include many more or less components than those shown in FIG. 14. However, the components shown are sufficient to disclose an illustrative embodiment for implementing the present disclosure. Client device 1400 may represent, for example, UE 102 discussed above at least in relation to FIG. 1.

As shown in the figure, in some embodiments, Client device 1400 includes a processing unit (CPU) 1422 in communication with a mass memory 1430 via a bus 1424. Client device 1400 also includes a power supply 1426, one or more network interfaces 1450, an audio interface 1452, a display 1454, a keypad 1456, an illuminator 1458, an input/output interface 1460, a haptic interface 1462, an optional global positioning systems (GPS) receiver 1464 and a camera(s) or other optical, thermal or electromagnetic sensors 1466. Device 1400 can include one camera/sensor 1466, or a plurality of cameras/sensors 1466, as understood by those of skill in the art. Power supply 1426 provides power to Client device 1400.

Client device 1400 may optionally communicate with a base station (not shown), or directly with another computing device. In some embodiments, network interface 1450 is sometimes known as a transceiver, transceiving device, or network interface card (NIC).

Audio interface 1452 is arranged to produce and receive audio signals such as the sound of a human voice in some embodiments. Display 1454 may be a liquid crystal display (LCD), gas plasma, light emitting diode (LED), or any other type of display used with a computing device. Display 1454 may also include a touch sensitive screen arranged to receive input from an object such as a stylus or a digit from a human hand.

Keypad 1456 may include any input device arranged to receive input from a user. Illuminator 1458 may provide a status indication and/or provide light.

Client device 1400 also includes input/output interface 1460 for communicating with external. Input/output interface 1460 can utilize one or more communication technologies, such as USB, infrared, Bluetooth™, or the like in some embodiments. Haptic interface 1462 is arranged to provide tactile feedback to a user of the client device.

Optional GPS transceiver 1464 can determine the physical coordinates of Client device 1400 on the surface of the Earth, which typically outputs a location as latitude and longitude values. GPS transceiver 1464 can also employ other geo-positioning mechanisms, including, but not limited to, triangulation, assisted GPS (AGPS), E-OTD, CI, SAI, ETA, BSS or the like, to further determine the physical location of Client device 1400 on the surface of the Earth. In one embodiment, however, Client device may through other components, provide other information that may be employed to determine a physical location of the device, including for example, a MAC address, Internet Protocol (IP) address, or the like.

Mass memory 1430 includes a RAM 1432, a ROM 1434, and other storage means. Mass memory 1430 illustrates another example of computer storage media for storage of information such as computer readable instructions, data structures, program modules or other data. Mass memory 1430 stores a basic input/output system (“BIOS”) 1440 for controlling low-level operation of Client device 1400. The mass memory also stores an operating system 1441 for controlling the operation of Client device 1400.

Memory 1430 further includes one or more data stores, which can be utilized by Client device 1400 to store, among other things, applications 1442 and/or other information or data. For example, data stores may be employed to store information that describes various capabilities of Client device 1400. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 1400.

Applications 1442 may include computer executable instructions which, when executed by Client device 1400, transmit, receive, and/or otherwise process audio, video, images, and enable telecommunication with a server and/or another user of another client device. Applications 1442 may further include a client 1445 that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising;
identifying, by a device, a set of categories corresponding to electronic activity data associated with a real-world location;
identifying, by the device, a set of data for each category in the set of categories;
analyzing, by the device, each set of data for each category;
determining, by the device, based on the analysis, a specific type of computational analysis to be performed for each set of data;
executing, by the device, each type of computational analysis;
determining, by the device, an electronic output for each category respectively based on the execution of each type of computational analysis;
generating, by the device, an interactive interface card for each determined output, the interactive interface card comprising functionality for facilitating communication among resources at the real-world location; and
causing display, by the device, over a network, of a displayable user interface (UI), the UI comprising functionality for displaying each generated interactive interface card.

2. The method of claim 1, further comprising:
monitoring, over the network, for updated information related to at least one category of the set of categories;
detecting the updated information for at least one category;
re-performing the computational analysis for the at least one category based on the updated information; and
dynamically updating the displayed interactive interface card for the at least one category.

3. The method of claim 1, further comprising:
detecting, over the network, an interaction with a displayed interactive interface card; and
causing a modification of the displayed UI, the modified UI displaying additional information related to the interacted with interface card.

4. The method of claim 3, wherein the modified UI comprises a newly displayed UI.

5. The method of claim 3, wherein the modified UI comprises a sub-window that enables interaction with at least one of the additional information, other users and functionality for providing feedback related to the category.

6. The method of claim 1, further comprising:
tracking progress of a task at the real-world location, wherein the tracked progress is enabled via at least one of a sensor or camera at the real-world location;
analyzing data related to the tracked progress;
determining, based on the analysis of the tracked progress, a status of the tasks; and
communicating the status to an account of a user, account having associated credentials enabling access to an interactive interface card for a user performing the task.

7. The method of claim 1, wherein the real-world location corresponds to a healthcare facility.

8. A method comprising;
identifying, by a device, a set of categories corresponding to electronic activity data associated with a real-world location;
identifying, by the device, a set of data for each category in the set of categories;
analyzing, by the device executing software defined by an artificial intelligence (AI) model, each set of data for each category;
determining, by the device, based on the AI model-based analysis, a specific type of computational analysis to be performed for each set of data;
executing, by the device, each type of computational analysis, the execution comprising accessing software code for each type of computational analysis and executing the accessed software code;
determining, by the device, an electronic output for each category respectively based on the execution of each type of computational analysis;
generating, by the device, an interactive interface card for each determined output, the interactive interface card comprising functionality for facilitating communication among resources at the real-world location; and
causing display, by the device, over a network, of a displayable user interface (UI), the UI comprising functionality for displaying each generated interactive interface card.

9. The method of claim 8, further comprising:
monitoring, over the network, for updated information related to at least one category of the set of categories;
detecting the updated information for at least one category;
re-performing the computational analysis for the at least one category based on the updated information; and
dynamically updating the displayed interactive interface card for the at least one category.

10. The method of claim 8, further comprising:
detecting, over the network, an interaction with a displayed interactive interface card; and
causing a modification of the displayed UI, the modified UI displaying additional information related to the interacted with interface card.

11. The method of claim 10, wherein the modified UI comprises a newly displayed UI.

12. The method of claim 10, wherein the modified UI comprises a sub-window that enables interaction with at least one of the additional information, other users and functionality for providing feedback related to the category.

13. The method of claim 8, wherein the AI model comprises at least one of a machine learning model and a large language model.

14. The method of claim 8, further comprising:
tracking progress of a task at the real-world location, wherein the tracked progress is enabled via at least one of a sensor or camera at the real-world location;
analyzing data related to the tracked progress;
determining, based on the analysis of the tracked progress, a status of the tasks; and
communicating the status to an account of a user, account having associated credentials enabling access to an interactive interface card for a user performing the task.

15. The method of claim 8, wherein the real-world location corresponds to a healthcare facility.

16. A system comprising:
a processor configured to:
identify a set of categories corresponding to electronic activity data associated with a real-world location;
identify a set of data for each category in the set of categories;
analyze each set of data for each category;
determine, based on the analysis, a specific type of computational analysis to be performed for each set of data;
execute each type of computational analysis;
determine an electronic output for each category respectively based on the execution of each type of computational analysis;
generate an interactive interface card for each determined output, the interactive interface card comprising functionality for facilitating communication among resources at the real-world location; and
cause display, over a network, of a displayable user interface (UI), the UI comprising functionality for displaying each generated interactive interface card.

17. The system of claim 16, wherein the processor is further configured to:
monitor, over the network, for updated information related to at least one category of the set of categories;
detect the updated information for at least one category;
re-perform the computational analysis for the at least one category based on the updated information; and
dynamically update the displayed interactive interface card for the at least one category.

18. The system of claim 16, wherein the processor is further configured to:
detect, over the network, an interaction with a displayed interactive interface card; and
cause a modification of the displayed UI, the modified UI displaying additional information related to the interacted with interface card.

19. The system of claim 18, wherein the modified UI comprises a sub-window that enables interaction with at least one of the additional information, other users and functionality for providing feedback related to the category.

20. The system of claim 16, wherein the processor is further configured to:
track progress of a task at the real-world location, wherein the tracked progress is enabled via at least one of a sensor or camera at the real-world location; analyzing data related to the tracked progress;
determining, based on the analysis of the tracked progress, a status of the tasks; and
communicating the status to an account of a user, account having associated credentials enabling access to an interactive interface card for a user performing the task.
